(19) United States
(12) Patent Application Publication
(10) Pub. No.: US 2024/0425891 A1
(43) Pub. Date: Dec. 26, 2024

(54) Host cells and methods for improved polyketide production using methylmalonyl-CoA-dependent polyketide synthase (PKS)

(71) Applicant: THE REGENTS OF THE UNIVERSITY OF CALIFORNIA, Oakland, CA (US)

(72) Inventors: Robert W. Haushalter, Emeryville, CA (US); Chunjun Zhan, Berkeley, CA (US); Jay D. Keasling, Berkeley, CA (US)

(21) Appl. No.: 18/751,141

(22) Filed: Jun. 21, 2024

Related U.S. Application Data
(60) Provisional application No. 63/509,712, filed on Jun. 22, 2023.
(60) Provisional application No. 63/593,853, filed on Oct. 27, 2023.

(51) Int. Cl. C12P 17/06; C12N 9/10

(52) U.S. Cl. CPC C12P 17/06; C12N 9/1025; C12N 9/1029; C12Y 203/03001

(57) ABSTRACT

The present invention provides for a genetically modified host cell reduced for expression of one of more endogenous enzyme(s) which enable the catabolism of propionyl-CoA and/or methylmalonyl-CoA, and comprising a methylmalonyl-CoA-dependent polyketide synthase (PKS).

### CROSS-REFERENCE TO RELATED APPLICATIONS

This application claims priority to U.S. Provisional patent application Ser. Nos. 63/509,712, filed Jun. 22, 2023, and 63/593,853, filed Oct. 27, 2023, which are hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of the production of compounds using a polyketide synthase.

### BACKGROUND OF THE INVENTION

Polyketides are a remarkable class of natural secondary metabolites produced by bacteria¹, fungi^{2, 3} and plants⁴. These natural products have drawn interest for decades owing to their valuable medicinal activities including antibiotic^{5, 6}, anticancer⁷, antifungal⁸, antiparasitic⁹ and immunosuppressive¹⁰ properties. Polyketide biosynthesis initiates with a variety of starter molecules, which are subsequently extended using malonyl-CoA, methylmalonyl-CoA, or other malonyl-CoA derivatives. Recent sequence analyses revealed that 55% of acyltransferases (AT) in known PKSs are malonyl-CoA specific and 30% are methylmalonyl-CoA specific¹¹, which indicates that malonyl-CoA and methylmalonyl-CoA are the key precursors for polyketide production. Malonyl-CoA serves as the basic precursor for the biosynthesis of many cellular building blocks (e.g., fatty acids) and a variety of secondary metabolites. Many strategies, including supplementation with acetate¹² and knockout competitive pathwayl³, have been adopted to increase malonyl-CoA concentration¹⁴. Otherwise, even though methylmalonyl-CoA is another crucial precursor for many bioproducts, it is not universally present in bacterial hosts 15.

Early work on expressing PKSs in heterologous hosts (e.g., Escherichia coli) explored several pathways to produce methylmalonyl-CoA¹⁶: addition of propionate and carboxylation of propionyl-CoA to methylmalonyl-CoA by propionyl-CoA carboxylase (PCCase), addition of succinate and isomerization to methylmalonyl-CoA by a heterologous mutase and epimerase¹⁷, and addition of methylmalonate and activation by a heterologous CoA ligase MatB. In E. coli, it was found that propionate feeding resulted in the highest titers of polyketide products (Murli et al. 2003; Boghigian et al. 2011). Analysis of carbon flux in these pathways shows that compared with other common carbon sources like glucose, the propionate carboxylation pathway also results in less carbon loss to CO2 (Extended Data FIG. S1; all Extended Data and Supplemental Information, including Extended Data Figures are available in the webpage for: nature.com/articles/s42255-023-00830-x), which is advantageous for production of commodity chemicals. All of these indicated that propionate may be a promising carbon source for methylmalonyl-CoA production.

Previously reported polyketides production primarily utilized Streptomyces as a host²¹⁻²³, which have the native propionate utilization pathway²⁰. However, the slow growth rate, complex life cycles, and relative lack of high efficiency genetic engineering tools^{24, 25} are major challenges for industrial scale-up of Streptomyces for production of commodity chemicals. C. glutamicum, an organism used to produce millions of tons of amino acids each year (Sano 2009), has many features that make it a promising host for polyketide production. This industrial microbe can natively produce methylmalonyl-CoA through one of its native propionate utilization pathways^{26, 27}, has many genetic tools available^{28, 29}, and harbors native PKSs for production of specialized lipids³⁰, further, it is closely related to Mycobacteria and Streptomyces³¹, which are rich sources of polyketide synthase biosynthetic gene clusters, all of which indicate that it might be a good host for heterologous PKSs. Addition of propionate to CGXII minimal media inhibits the growth of C. glutamicum, but the metabolic basis of this inhibition was unclear prior to our work. C. glutamicum encodes two distinct pathways for catabolizing propionate. In the 2-methylcitrate pathway, propionate is oxidized to pyruvate while oxaloacetate is reduced to succinate via the intermediate 2-methylcitrate. This pathway is essential for growth on propionate as the sole carbon source on minimal media26. In the methylmalonyl-CoA pathway, an endogenous carboxylase converts propionyl-CoA to(S)-methylmalonyl-CoA, which is then epimerized to (R)-methylmalonyl-CoA by the enzyme encoded by cgl1217, and isomerized to succinyl-CoA by the vitamin B12-dependent mutase (McmAB) (FIG. 1).

Previous reports have suggested that accumulation of 2-methylcitrate is the cause of inhibition (Plassmeier et al. 2007), but we show here that propionate addition still inhibits growth when this pathway is removed. It has been shown that addition of vitamin B12, the required cofactor for McmAB, relieves growth inhibition in the presence of propionate (Botella et al. 2009). This led us to the hypothesis that accumulation of propionyl-CoA and methylmalonyl-CoA is causing growth inhibition, and pathways that convert methylmalonyl-CoA to free CoA-SH or another primary metabolite will impart a growth advantage to C. glutamicum in propionate media.

### SUMMARY OF THE INVENTION

The present invention provides for a genetically modified host cell reduced for expression of one of more endogenous enzyme(s) which enable the catabolism of propionyl-CoA and/or methylmalonyl-CoA, and comprising a methylmalonyl-CoA-dependent polyketide synthase (PKS). The methylmalonyl-CoA-dependent PKS is a PKS that uses methylmalonyl-CoA as substrate.

In some embodiments, the genetically modified host cell is a genetically modified Corynebacterium cell. In some embodiments, the genetically modified Corynebacterium cell is a Corynebacterium glutamicum. In some embodiments, the methylmalonyl-CoA-dependent polyketide synthase is a hybrid PKS.

In some embodiments, the hybrid PKS comprises a loading domain capable of loading methylmalonyl-CoA as a starting substrate. In some embodiments, the loading domain capable of loading methylmalonyl-CoA as a starting substrate is the loading domain of germicidin PKS. Germicidin synthase (Gcs) from Streptomyces coelicolor is a type III polyketide synthase (PKS) with broad substrate flexibility for acyl groups linked through a thioester bond to either coenzyme A (CoA) or acyl carrier protein (ACP). In some embodiments, methylmalonyl-CoA-dependent production of germicidin and 3-hydroxyacids rescue cell growth in propionate-containing medium.

A system comprising the genetically modified Corynebacterium cell of the present invention.

The present invention provides for a genetically modified Corynebacterium cell, such a Corynebacterium glutamicum, can be used as a host cell for improved polyketide production. In some embodiments, the system comprises the genetically modified Corynebacterium cell comprising the genes which enable catabolism of propionyl-CoA and methylmalonyl-CoA deleted or knocked out, and genes encoding methylmalonyl-CoA-dependent polyketide synthases are integrated. In some embodiments, the genetically modified Corynebacterium cell with greater polyketide production grows faster or have a shorter doubling or generation time. This enables applications for improving polyketide production in using host cells by 1) creating mutant libraries of PKS genes and using this system to screen for higher producers, and/or 2) performing directed evolution experiments to generate and screen for productive mutations in other loci of the host genome. In some embodiments, the system improves the production of Germicidin in C. glutamicum. In some embodiments, the system can be generalized to any acyl-CoA species which can accumulate to levels that inhibit growth.

In some embodiments, the genetically modified host cell comprises: 1) removal of metabolic pathway genes that enable catabolism or breakdown of an acyl-CoA, and 2) introduction of a biosynthetic pathway which uses said acyl-CoA as a substrate. When host cells with these genetic modifications are grown in the presence of media components that lead to accumulation of an acyl-CoA, the cells which have the most active biosynthetic pathway will consume the most acyl-CoA and grow faster. This enables screening and selection for mutant cells which have improved polyketide production. In some embodiments, the genetically modified host cell is reduced in expression, knocked out or deleted for one or more, of all, genes encoding for PrpC, PrpD, PrpB, MutaseA, and/or MutaseB. In some embodiments, the gene reduced in expression, knocked out or deleted is a gene endogenous to the host cell. In some embodiments, the genetically modified host cell is overexpressed for one or more, of all, genes encoding for AcpA and/or FabH. In some embodiments, the overexpressed gene is endogenous to the host cell, or is a gene heterologous to the host cell. In some embodiments, the overexpressed gene is operatively linked to a promoter. In some embodiments, the promoter is constitutive or inducible. In some embodiments, the promoter is heterologous to the host cell and/or the gene. In some embodiments, there are multiple copies of the gene that is overexpressed.

In some embodiments, the genetically modified host cell is a Corynebacterium glutamicum constructed by: 1) removing one or more metabolic pathway genes that enable catabolism of propionyl-CoA and methylmalonyl-CoA and 2) introduction of the Germicidin biosynthetic pathway which uses methylmalonyl-CoA as a substrate. The cells in the presence of propionate (which leads to accumulation of methylmalonyl-CoA), and selected mutant strains which grew faster than the original strain after multiple generations of growth in propionate. It is demonstrated that one can select mutant strains with improved production of germicidin compared to strains which were not generated using the invention.

The present system can be used to isolate several improved polyketide production strains using propionate and a methylmalonyl-CoA dependent pathway. This invention can be used to improve the production of any natural product which uses methylmalonyl-CoA as a substrate. This includes many high-value natural products such as erythromycin (antibiotic) and spinosyn (insecticide). This system could also be used to increase the production from engineered polyketide synthases which use methylmalonyl-CoA for the production of novel recyclable materials, flavors and fragrances, or other commodity or specialty chemicals.

In some embodiments, the present invention comprises a method producing a polyketide using this modified host cell in a media supplementation with an acyl-CoA precursor leads to growth inhibition which can be leveraged as a selection strategy for improved production. Previously reported host cells, including E. coli and Streptomyces species, are not growth inhibited by propionate like C. glutamicum, and therefore they cannot be used as a selective tool.

The present invention provides for a method of producing a polyketide using methylmalonyl-CoA as a substrate, said method comprising: (a) providing a genetically modified host cell of the present invention, and (b) culturing or growing the genetically modified host cell such that the genetically modified host cell expresses the methylmalonyl-CoA-dependent PKS and produces a polyketide using methylmalonyl-CoA as a substrate.

In some embodiments, the growing or culturing step of the method uses a media comprising a renewable carbon source, such as lignocellulosic biomass. In some embodiments, the lactam compound is produced using a one-pot pretreatment saccharification and fermentation process. In some embodiments, the nucleic acid encoding each enzyme described herein is codon optimized to the genetically modified host cell.

In some embodiments, the culturing or growing step (b) comprises the host cell growing by respiratory cell growth. In some embodiments, the culturing or growing step (b) takes place in a batch process or a fed-batch process, such as a high-gravity fed-batch process. In some embodiments, the culture comprises a biomass, such as a lignocellulosic biomass, or hydrolysate thereof. In some embodiments, the biomass is obtained from softwood feedstock (such as poplar), hardwood feedstock, grass feedstock, and/or agricultural feedstock, or mixture thereof.

In some embodiments, the culture or medium comprises a rich medium, such as LB (Lysogeny-Broth) or comprising one or more ingredients of LB, such as tryptone and/or yeast extract. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable. In some embodiments, the culture or medium comprises urea as a nitrogen source. In some embodiments, the culture or medium comprises an ionic liquid (IL).

In some embodiments, the invention comprises the use of a heterologous codon-optimized version of each nucleic acid encoding the described enzyme, which are optimized to the genetically modified host cell.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Schematic showing the overall propionate metabolism in C. glutamicum. We used a function-based strategy to determine the metabolic basis of propionate induced growth inhibition, and used adaptive laboratory evolution to improve polyketide titers. Our engineering focused on three different parts. Part I (Pink) is the pathway providing propionate uptake and activation to propionyl-CoA. Part II (Green) is the pathway involved in propionyl-CoA assimilation and conversion to primary metabolites. C. glutamicum encodes both a methylcitrate pathway and a methylmalonyl-CoA pathway. Part III (Blue) is methylmalonyl-CoA-dependent PKSs that were tested to consume methylmalonyl-CoA and relieve growth inhibition. The black line represents the native pathways, the orange line represents the enhanced pathways and the gray line represents the knockout pathways.

FIG. 2 Propionate derivatives induce growth inhibition. a: Two pathways for converting propionate to propionyl-CoA. b: Growth curves of wild-type and engineered propionate uptake strains in CGXII minimal medium with 1,000 mg/L propionate: Cz01 (Δptaack), Cz02 (Δcgl2569), C203 (Δcgl2569Δptaack). c: Propionate consumed by the four strains. d: Growth curves of wild-type and Cg12569 overexpressed strains in CGXII minimal medium with 1,000 mg/L propionate: Cz19 (overexpress Cg12569 in wild-type), Cz22 (overexpress Cg12569 in Cz02). c: Intracellular propionyl-CoA and methylmalonyl-CoA concentrations in the four strains. All strains were pre-cultivated in BHI medium overnight (30° C.), then cells were transformed into CGXII minimal medium, 30° C. for 16 h. Finally, cells were transformed into fresh CGXII minimal medium with different concentrations of propionate. All data represents the mean±SD, and error bars indicate the standard error (n=3), *p<0.05; **p<0.05; ***p<0.05.

FIG. 3 Accumulation of propionyl-CoA and methylmalonyl-CoA induces growth inhibition in propionate media. a: propionyl-CoA metabolism includes two pathways: (i) 2-methylcitrate pathway (pink) and (ii) methylmalonyl-CoA pathway (orange and light blue). b: Growth curve of various strains in CGXII minimal medium with 1,000 mg/L propionate. c: Growth curves of different mutants in CGXII minimal medium with 1,000 mg/L propionate and 10 μM vitamin B12. d: Growth curve of strains overexpressing prpE and PCCase grown in CGXII minimal medium with 1,000 mg/L propionate. e: Growth curve of CoA-engineered strains in CGXII minimal medium with 600 mg/L propionate. Cz04 (ΔmcmAB), Cz05 (ΔprpDBC1/2), Cz06 (ΔmcmAB, ΔprpDBC1/2), Cz07 (ΔprpDBC1/2:prpE), Cz08 (cglp3:pcc), Cz09 (ΔprpDBC1/2:prpE, cglp3:pcc), Cz10 (ΔprpDBC1/2:aspA, cglp3:panD). Cells were precultivated in BHI medium overnight (30° C.), then cells were subcultured into CGXII medium (200 rpm, 30° C.) and grown for 16 h. Finally, different strains were cultivated in CGXII+1,000 mg/L propionate (200 rpm, 30° C.). All data represent the mean±SD, and error bars indicate the standard error (n=3).\

FIG. 4 Methylmalonyl-CoA dependent Germicidin production can rescue cell growth in propionate medium. a: Scheme of different methylmalonyl-CoA dependent PKSs production pathways. b: Growth curves of different strains in CGXII minimal medium with 600 mg/L propionate. Cz34 (ΔmcmAB, ΔprpDB (1, prpDBC2:sfp), Cz11 (Cz34 cglp3:rfp), Cz12 (integrate whole germicidin pathway genes in Cz34) c: Lag phase comparison of strains containing various PKSs in CGXII minimal medium with 600 mg/L propionate. Cz26 (integrate LipLM-M1-debsM6TE in Cz34), Cz27 (integrate type III PKS 2470 in Cz34), Cz28 (integrate type III PKS 2474 in Cz34). d: Germicidin C titer and lag phase in Cz12 strain cultivated at different concentrations of propionate. All data represent the mean±SD, and error bars indicate the standard error (n=3).

FIG. 5 Evolved germicidin production strains show increased titer and improved growth. a: Growth curves of Cz34 (no germicidin pathway), Cz12 (germicidin pathway integrated), and CzEv208 (single colony from Cz13 evolved population strains) in CGXII minimal medium with 8,000 mg/L propionate. For comparison, we included the growth curve of WT in CGXII minimal medium without propionate. b: Germicidin production comparison among Cz12 and 20 evolved strains. c: Modeled surface structure of Gcs based on module 3v7i.1.A (PDB). Residues which were mutated in evolved strains are labeled. Pink represents mutation sites L216P, T257A and R347L. Active site catalytic triad residues Cys175-His312-Asn346 are shown in green. Cyan represents four Arg (276/277/280/317) residues adjacent to the catalytic triad active site35. d: Growth curve and Germicidin titer among different reversed engineering strains. All strains were pre-cultivated in BHI medium overnight (30° C.), and then inoculated in CGXII minimal medium (30° C. for 16 h), then cells were transferred into fresh CGXII medium with 8,000 mg/L propionate. Cz12 (integrate germicidin pathway in Cz34), CzEv208 (single colony from evolved strains), Cz32 (replace evolved gcs with native gcs in CzEv208), Cz33 (replace native gcs with Gcs R347L in Cz12). All data represent the mean±SD, and error bars indicate the standard error (n=3).

FIG. 6 Propionyl-CoA was converted into germicidin by GltA2-based methylcitrate pathway. a: Clusters of orthologous groups (COG) analysis (Huerta-Cepas et al. 2019). COG category of C. glutamicum genes were screened by using EggNOG (Huerta-Cepas, J. et al. eggNOG 5.0: a hierarchical, functionally and phylogenetically annotated orthology resource based on 5090 organisms and 2502 viruses. Nucleic Acids Res 47, D309-D314 (2019)) and COG category distribution of SNP containing genes were compared with that of total genes. Right panel is the proportion of strains having SNPs in the corresponding genes assigned to COG category C and G among all 19 evolved strains. b: Growth curves of strains with specific gltA mutations grown in CGXII minimal medium with 8 g/L propionate. c: In vitro citrate synthase assay using purified GltA2 proteins. Acetyl-CoA and/or propionyl-CoA are consumed in the reaction, releasing free CoA-SH which is measured colorimetrically. d: Growth curves of different gltA2 based strains. e: GltA2-based methylcitrate pathway, orange line represents [13C] labeled carbon. All strains were pre-cultivated in BHI medium overnight (30° C.), and then transformed into CGXII minimal medium (30° C. for 16 h), then cells were transferred into fresh CGXII medium with 8,000 mg/L propionate. All data represent the mean±SD, and error bars indicate the standard error (n=3).

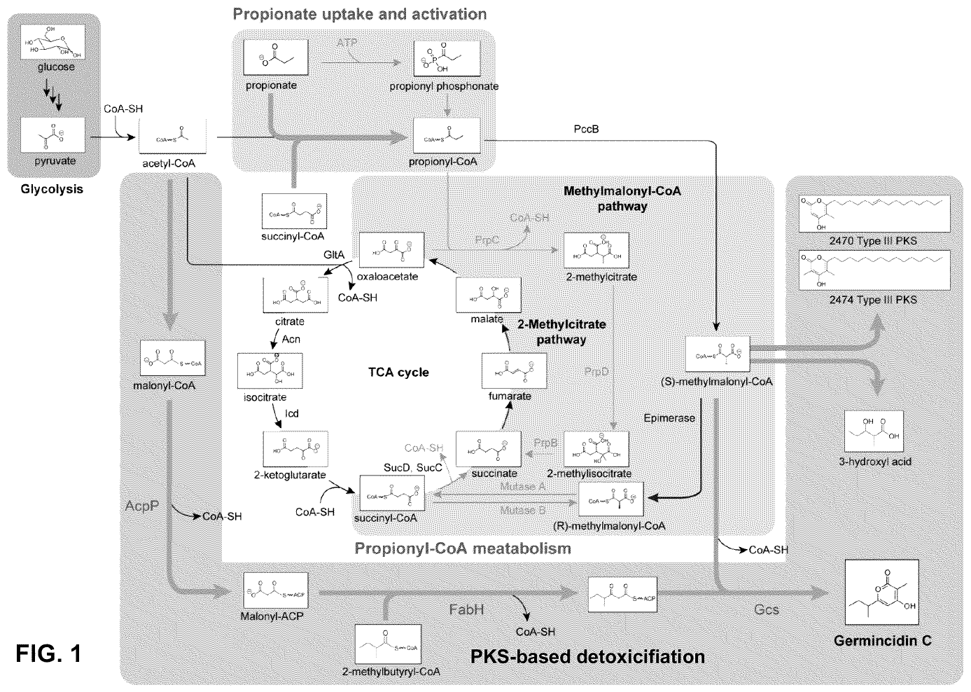

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “host cell” and “host microorganism” are used interchangeably herein to refer to a living biological cell, such as a microbe, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to a host cell or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

### Enzymes, and Nucleic Acids Encoding Thereof

A homologous enzyme is an enzyme that has a polypeptide sequence that is at least 70%, 75%, 80%, 85%, 90%, 95% or 99% identical to any one of the enzymes described in this specification or in an incorporated reference. The homologous enzyme comprises or retains amino acid residues that are recognized as conserved for the enzyme. The homologous enzyme may have non-conserved amino acid residues replaced or found to be of a different amino acid, or amino acid(s) inserted or deleted, but which does not affect or has insignificant effect on the enzymatic activity of the homologous enzyme. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject enzymes. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, Xhol, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host microorganism simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the lactam compound ensured. When added, any intermediate is present in an excess amount in the culture medium.

Any means for extracting or separating the lactam compound from the host cell may be used. For example, the host cell may be harvested and subjected to hypotonic conditions, thereby lysing the cells. The lysate may then be centrifuged and the supernatant subjected to high performance liquid chromatography (HPLC) or gas chromatography (GC).

### Host Cells

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

Each introduced enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

The genetically modified host cell can be any bacterial cell capable of production of the lactam compound of the present invention in accordance with the methods of the invention.

In some embodiments, the host cell is a prokaryotic cell, such as a bacterial cell. In some embodiments, the host cell is a bacterial cell selected from the Escherichia, Enterobacter, Azotobacter, Erwinia, Bacillus, Pseudomonas, Klebsiella, Proteus, Salmonella, Serratia, Shigella, Ralstonia, Rhizobia, or Vitreoscilla taxonomical class. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, B. pumilus, B. brevis, B. aminovorans, or B. fusiformis. In some embodiments the bacterial cell is a Gram-positive bacterium, such as a Streptomyces species, such as any Streptomyces species or strain taught herein.

The genetically modified host cell can be any yeast capable of production of the lactam compound in accordance with the methods of the invention.

In some embodiments, the host cell is a yeast. Yeast host cells suitable for the invention include, but are not limited to, Yarrowia, Candida, Bebaromyces, Saccharomyces, Schizosaccharomyces and Pichia cells. In one embodiment, Saccharomyces cerevisae is the host cell. In one embodiment, the yeast host cell is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In one embodiment, Candida tropicalis is the host cell.

In some embodiments, the yeast host cell is a non-oleaginous yeast. In some embodiments, the yeast host cell is a basidiomycete. In some embodiments, the yeast host cell is an oleaginous yeast. In some embodiments, the oleaginous yeast is a Rhodosporidium species. In some embodiments, the Rhodosporidium species is Rhodosporidium toruloides. In some embodiments, the Rhodosporidium toruloides is strain IFO 0880.

In some embodiments, the host cell comprises a nucleic acid encoding the one or more enzymes operatively linked to a promoter capable of expressing the one or more enzymes in the host cell. In some embodiments, the encoding of the one or more enzymes to the nucleic acid is codon optimized to the host cell. In some embodiments, the nucleic acid is vector or replicon that can stably reside in the host cell. In some embodiments, the nucleic acid is stably integrated into one or more chromosomes of the host cell.

In some embodiments, the providing step (a) comprises introducing a nucleic acid encoding the one or more enzymes operatively linked to a promoter capable of expressing the one or more enzymes in the host cell into the host cell.

In some embodiments, the culturing or growing step (b) comprises the host cell growing by respiratory cell growth. In some embodiments, the culturing or growing step (b) takes place in a batch process or a fed-batch process, such as a high-gravity fed-batch process. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable.

The present invention provides for a method for constructing a genetically modified host cell of the present invention, comprising (a) introducing a nucleic acid encoding the one or more enzymes operatively linked to a promoter capable of expressing the one or more enzymes in the host cell into the host cell.

One can modify the expression of a gene encoding any of the enzymes taught herein by a variety of methods in accordance with the methods of the invention. Those skilled in the art would recognize that increasing gene copy number, ribosome binding site strength, promoter strength, and various transcriptional regulators can be employed to alter an enzyme expression level.

### Suitable Biomass

The biomass can be obtained from one or more feedstock, such as softwood feedstock, hardwood feedstock, grass feedstock, and/or agricultural feedstock, or a mixture thereof.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus sempervirene); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus tacda, Pinus palustris, Pinus rigida, Pinus echinata); Redwood; Rimu; Spruce (e.g. Picea abies, Picea mariana, Picea rubens, Picca sitchensis, Picca glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus tacda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ipê; Iroko; Ironwood (e.g. Bangkirai, Carpinus caroliniana, Casuarina equisetifolia, Choricbangarpia subargentea, Copaifera spp., Eusideroxylon zwageri, Guaiacum officinale, Guaiacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacaranda; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoumé; Oregon Myrtle; California Bay Laurel; Pear; Poplar (e.g. P. balsamifera, P. nigra, Hybrid Poplar (Populus canadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C4 or C3 grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, rice, soybeans, hay, potatoes, cotton, or sugarcane.

The feedstock can arise from the harvesting of crops from the following practices: intercropping, mixed intercropping, row cropping, relay cropping, and the like.

Other objects, features, and advantages of the present invention will be apparent to one of skill in the art from the following detailed description and figures.

### References Cited Herein:

1. Moore, B. S. & Hopke, J. N. Discovery of a new bacterial polyketide biosynthetic pathway. Chembiochem 2, 35-38 (2001).
2. Smith, C. J. et al. Novel polyketide metabolites from a species of marine fungi. J Nat Prod 63, 142-145 (2000).
3. Mapari, S. A. et al. Exploring fungal biodiversity for the production of water-soluble pigments as potential natural food colorants. Curr Opin Biotechnol 16, 231-238 (2005).
4. Flores-Sanchez, I. J. & Verpoorte, R. Plant polyketide synthases: a fascinating group of enzymes. Plant Physiol Biochem 47, 167-174 (2009).
5. Doull, J. L., Ayer, S. W., Singh, A. K. & Thibault, P. Production of a novel polyketide antibiotic, jadomycin B, by Streptomyces venezuelae following heat shock. J Antibiot (Tokyo) 46, 869-871 (1993).
6. Bangera, M. G. & Thomashow, L. S. Identification and characterization of a gene cluster for synthesis of the polyketide antibiotic 2,4-diacetylphloroglucinol from Pseudomonas fluorescens Q2-87. J Bacteriol 181, 3155-3163 (1999).
7. Ratnayake, R., Lacey, E., Tennant, S., Gill, J. H. & Capon, R. J. Kibdelones: novel anticancer polyketides from a rare Australian actinomycete. Chemistry 13, 1610-1619 (2007).
8. Piao, S. J. et al. Hippolachnin A, a new antifungal polyketide from the South China Sea sponge Hippospongia lachne. Org Lett 15, 3526-3529 (2013).
9. Shi, Y. M. et al. Georatusin, a Specific Antiparasitic Polyketide-Peptide Hybrid from the Fungus Geomyces auratus. Org Lett 20, 1563-1567 (2018).
10. Hong, H., Demangel, C., Pidot, S. J., Leadlay, P. F. & Stinear, T. Mycolactones: immunosuppressive and cytotoxic polyketides produced by aquatic mycobacteria. Nat Prod Rep 25, 447-454 (2008).
11. Keatinge-Clay, A. T. The Uncommon Enzymology of Cis-Acyltransferase Assembly Lines. Chem Rev 117, 5334-5366 (2017).
12. Zha, W., Rubin-Pitel, S. B., Shao, Z. & Zhao, H. Improving cellular malonyl-CoA level in Escherichia coli via metabolic engineering. Metab Eng 11, 192-198 (2009).
13. Takamura, Y. & Nomura, G. Changes in the intracellular concentration of acetyl-CoA and malonyl-CoA in relation to the carbon and energy metabolism of Escherichia coli K12. J Gen Microbiol 134, 2249-2253 (1988).
14. Milke, L. & Marienhagen, J. Engineering intracellular malonyl-CoA availability in microbial hosts and its impact on polyketide and fatty acid synthesis. Appl Microbiol Biotechnol 104, 6057-6065 (2020).
15. Mattozzi, M., Ziesack, M., Voges, M. J., Silver, P. A. & Way, J. C. Expression of the sub-pathways of the Chloroflexus aurantiacus 3-hydroxypropionate carbon fixation bicycle in E. coli: Toward horizontal transfer of autotrophic growth. Metab Eng 16, 130-139 (2013).
16. Gonzalez-Garcia, R. A., Nielsen, L. K. & Marcellin, E. Heterologous production of 6-deoxyerythronolide B in Escherichia coli through the Wood Werkman Cycle. Metabolites 10, 228 (2020).
17. Haller, T., Buckel, T., Rétey, J. & Gerlt, J. A. Discovering new enzymes and metabolic pathways: conversion of succinate to propionate by Escherichia coli. Biochemistry 39, 4622-4629 (2000).
18. Enjalbert, B., Millard, P., Dinclaux, M., Portais, J. C. & Letisse, F. Acetate fluxes in Escherichia coli are determined by the thermodynamic control of the Pta-AckA pathway. Sci Rep 7, 42135 (2017).
19. Ingram-Smith, C. et al. Characterization of the acetate binding pocket in the Methanosarcina thermophila acetate kinase. J Bacteriol 187, 2386-2394 (2005).
20. Murli, S., Kennedy, J., Dayem, L. C., Carney, J. R. & Kealey, J. T. Metabolic engineering of Escherichia coli for improved 6-deoxyerythronolide B production. J Ind Microbiol Biotechnol 30, 500-509 (2003).
21. Yuzawa, S. et al. Short-chain ketone production by engineered polyketide synthases in Streptomyces albus. Nat Commun 9, 4569 (2018).
22. Risdian, C., Mozef, T. & Wink, J. Biosynthesis of Polyketides in Streptomyces. Microorganisms 7 (2019).
23. Cruz-Morales, P. et al. Biosynthesis of polycyclopropanated high energy biofuels. Joule 6, 1590-1605 (2022).
24. Weber, T., Welzel, K., Pelzer, S., Vente, A. & Wohlleben, W. Exploiting the genetic potential of polyketide producing streptomycetes. J Biotechnol 106, 221-232 (2003).
25. Lee, N. et al. Synthetic Biology Tools for Novel Secondary Metabolite Discovery in Streptomyces. J Microbiol Biotechnol 29, 667-686 (2019).
26. Claes, W. A., Puhler, A. & Kalinowski, J. Identification of two prpDBC gene clusters in Corynebacterium glutamicum and their involvement in propionate degradation via the 2-methylcitrate cycle. J Bacteriol 184, 2728-2739 (2002).
27. Huser, A. T. et al. Development of a Corynebacterium glutamicum DNA microarray and validation by genome-wide expression profiling during growth with propionate as carbon source. J Biotechnol 106, 269-286 (2003).
28. Kirchner, O. & Tauch, A. Tools for genetic engineering in the amino acid-producing bacterium Corynebacterium glutamicum. J Biotechnol 104, 287-299 (2003).
29. Nešvera, J. & Pátek, M. Tools for genetic manipulations in Corynebacterium glutamicum and their applications. Applied microbiology and biotechnology 90, 1641-1654 (2011).
30. Portevin, D. et al. A polyketide synthase catalyzes the last condensation step of mycolic acid biosynthesis in mycobacteria and related organisms. Proc Natl Acad Sci USA 101, 314-319 (2004).
31. Lee, J. Y., Park, J. S., Kim, H. J., Kim, Y. & Lee, H. S. Corynebacterium glutamicum whcB, a stationary phase-specific regulatory gene. FEMS Microbiol Lett 327, 103-109 (2012).
32. Veit, A. et al. Pathway identification combining metabolic flux and functional genomics analyses: acetate and propionate activation by Corynebacterium glutamicum. J Biotechnol 140, 75-83 (2009).
33. Botella, L., Lindley, N. D. & Eggeling, L. Formation and metabolism of methylmalonyl coenzyme A in Corynebacterium glutamicum. J Bacteriol 191, 2899-2901 (2009).
34. Park, Y. K., Ledesma-Amaro, R. & Nicaud, J. M. De novo Biosynthesis of Odd-Chain Fatty Acids in Yarrowia lipolytica Enabled by Modular Pathway Engineering. Front Bioeng Biotechnol 7, 484 (2019).
35. Chemler, J. A. et al. Biochemical and structural characterization of germicidin synthase: analysis of a type III polyketide synthase that employs acyl-ACP as a starter unit donor. J Am Chem Soc 134, 7359-7366 (2012).
36. Bai, Y. et al. Structure-based molecular networking for the target discovery of novel germicidin derivatives from the sponge-associated streptomyces sp. 18A01. J Antibiot (Tokyo) (2021).
37. Glaser, L. et al. A common approach for absolute quantification of short chain CoA thioesters in prokaryotic and eukaryotic microbes. Microb Cell Fact 19, 160 (2020).
38. Mullins, E. A., Francois, J. A. & Kappock, T. J. A specialized citric acid cycle requiring succinyl-coenzyme A (CoA): acetate CoA-transferase (AarC) confers acetic acid resistance on the acidophile Acetobacter aceti. J Bacteriol 190, 4933-4940 (2008).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Leveraging Propionate-Induced Growth Inhibition in Corynebacterium glutamicum to Evolve Improved Methylmalonyl-CoA-Dependent Polyketide Production

The Corynebacterium glutamicum is a promising host for production of valuable polyketides. Propionate addition, a strategy that is known to increase polyketide production by increasing methylmalonyl-CoA, causes growth inhibition in C. glutamicum. The mechanism of this inhibition was unclear prior to our work. Here we provide evidence that accumulation of propionyl- and methylmalonyl-CoA induces growth inhibition in C. glutamicum. We then showed that growth inhibition can be relieved by introducing methylmalonyl-CoA dependent polyketide synthases. Using germicidine as an example, we used adaptive laboratory evolution (ALE) to leverage the fitness advantage of polyketide production in the presence of propionate to evolve improved germicidin production. Whole genome sequencing revealed mutations in germicidin synthase (Gcs), which improved germicidin titer, as well as mutations in citrate synthase, which effectively evolved the native glyoxylate pathway to a new methylcitrate pathway. Together, our results show that C. glutamicum is not only a capable heterologous host for polyketide production, but we can take advantage of this organism's intrinsic inhibition by propionate to drive titers higher by directed evolution.

Here, we show that accumulation of propionyl- and methylmalonyl-CoA is the cause of growth inhibition by propionate in C. glutamicum, and blocking conversion of these CoAs to primary metabolites inhibits growth on propionate. Because conversion of methylmalonyl-CoA into succinyl-CoA was shown to relieve the propionate-induced growth lag, several methylmalonyl-CoA-consuming polyketide production pathways, including germicidin synthase (Gcs) and an engineered lipomycin synthase, were introduced to provide a means of reducing the intracellular methylmalonyl-CoA concentration. We found that polyketide production increase is associated with a shorter lag phase when grown in minimal media containing propionate. Furthermore, we demonstrated that addition of propionate can be used as a selective pressure for ALE of PKSs in C. glutamicum. Using ALE in the presence of propionate, we increased the titer of germicidin over 18-fold and thoroughly eliminated growth inhibition in propionate medium. Next generation sequencing (NGS) and reverse engineering results demonstrated that the evolved Gcs can improve germicidin production and the evolved citrate synthase (GltA2)-based methylcitrate pathway rescued cell growth by consuming propionyl-CoA and methylmalonyl-CoA. Taken together, our results show that C. glutamicum is not only capable of expressing PKSst, but also can be used as a platform to improve polyketide titers through directed evolution.

### Results

1. Propionate Induces Growth Inhibition when Activated to Propionyl-CoA

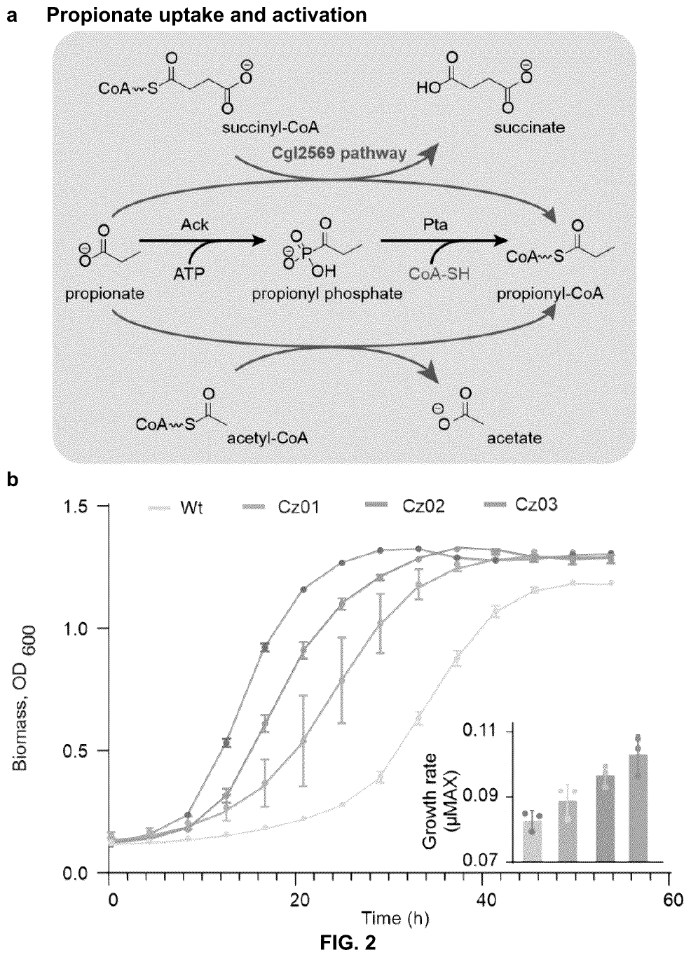

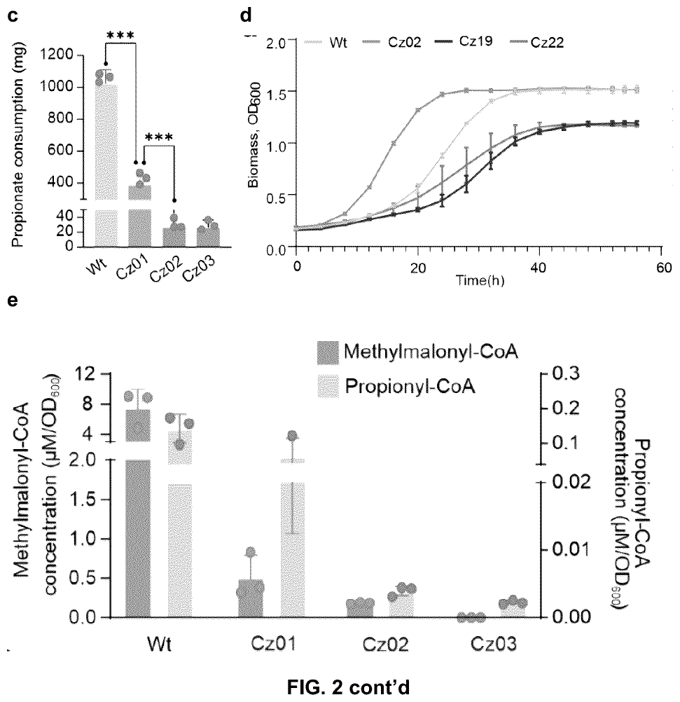

To determine whether inhibition is caused by free propionate or its metabolic derivatives, we first evaluated the propionate uptake and activation pathway (FIG. 2, panel a). C. glutamicum can activate propionate using the acetate uptake pathway, including acetate kinase (Ack) and phosphotransacetylase (Pta)²⁶. We deleted those two genes to generate strain Cz01. Compared with wild-type, the growth rate of Cz01 increased by 7% (FIG. 2, panel b), and propionate consumption and the lag phase decreased by 60% and 40%, respectively (FIG. 2, panel c and Extended Data FIG. 2). These results indicate that propionate must be activated to propionyl-CoA to induce growth inhibition. Besides the acetate uptake pathway, it has also been indicated that the CoA transferase (succinyl-CoA: acetate CoA-transferase) encoded by cgl2569 may be another propionate activation pathway³², catalyzing conversion of propionate to propionyl-CoA while succinate/acetate is produced by succinyl-CoA/acetyl-CoA consumption (FIG. 2, panel a). To validate whether this pathway contributes to propionate-induced growth inhibition, we deleted cgl2569 from the wild-type strain to generate strain Cz02, and evaluated its growth in propionate-containing medium. Cz02 grew faster (growth rate increased 25%), consumed less propionate (decreased by 99%) and had a shorter lag phase (decreased by 50%), compared to the wild-type strain (FIG. 2, panels b, c, and Extended Data FIG. S2). Additionally, we performed in vitro experiments using purified Cg12569 protein (Extended Data FIG. S3a). Liquid chromatography-mass spectrometry (LC-MS) results showed that Cg12569 can convert propionate to propionyl-CoA using succinyl-CoA/acetyl-CoA as CoA donor (Extended Data FIG. S3). Cg12569 was overexpressed in wild-type and C202 to generate Cz19 and Cz22, respectively. Results showed that cell growth was further inhibited when cgl2569 was overexpressed (FIG. 2, panel d), consistent with the reasoning that the inhibition mechanism involves propionyl-CoA. Deletion of cgl2569 particularly decreased the uptake of propionate (decreased by 99%) compared to deletion of the acetate uptake pathway (decreased by 60%) (FIG. 2, panels b, c), which indicates that the cgl2569 pathway is more active than the acetate uptake pathway for propionate activation in C. glutamicum. In addition to propionate consumption, measuring its metabolic derivatives, such as intracellular methylmalonyl-CoA and propionyl-CoA, directly showed the correlation between length of lag phase and concentrations of these CoAs (FIG. 2, panels a and c). Taken together, these results show that propionate derivatives like propionyl-CoA and methylmalonyl-CoA may be the cause of growth inhibition, not free propionate itself.

### 2. Methylmalonyl-CoA Accumulation Induces Growth Inhibition in Propionate Medium

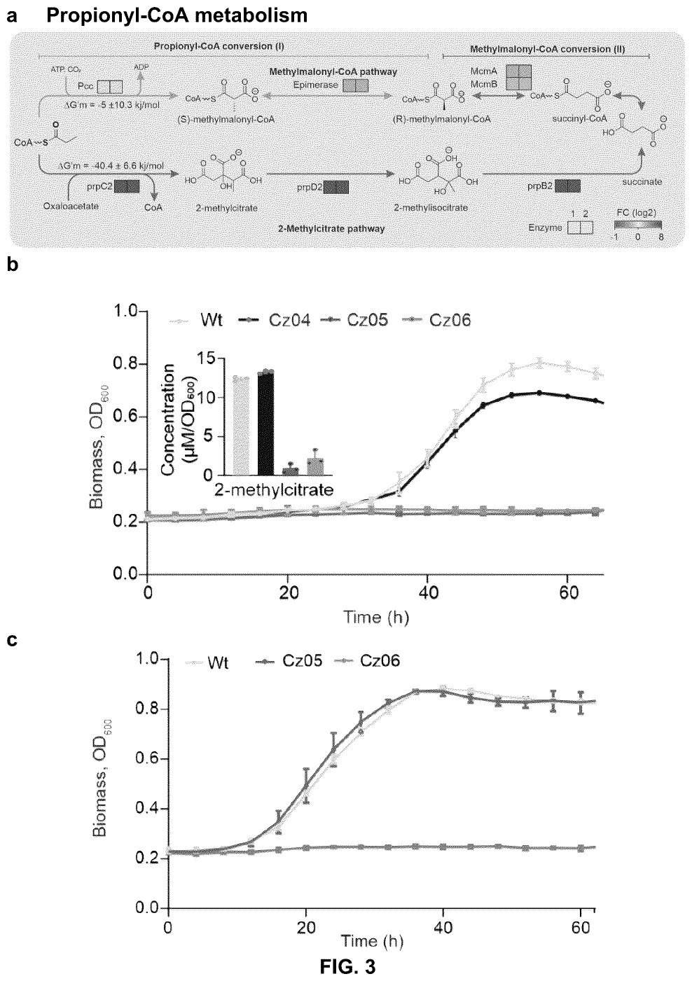

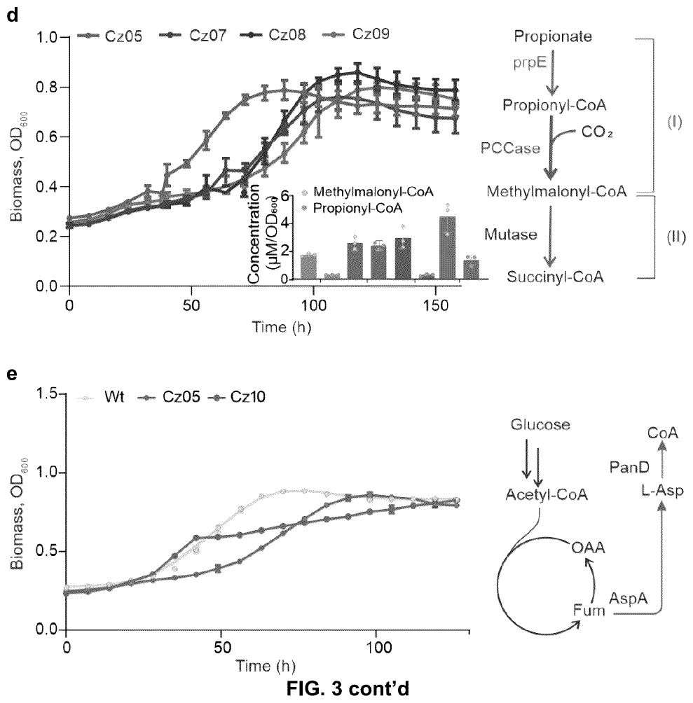

To determine the cause of the dose-dependent growth lag in propionate, we next evaluated the propionyl-CoA metabolism. C. glutamicum encodes two distinct pathways for propionyl-CoA metabolism. The first is the 2-methylcitrate pathway, in which propionyl-CoA is oxidized to pyruvate to release free CoA while oxaloacetate is reduced to succinate. An alternative is methylmalonyl-CoA pathway, which involves carboxylation of propionyl-CoA to yield(S)-methylmalonyl-CoA followed by isomerization to (R)-methylmalonyl-CoA, and then to the TCA cycle intermediate succinyl-CoA to support cell growth (FIG. 3, panel a) 26. Transcriptomic analysis showed that the three genes (prpD2B2C2) encoding the 2-methylcitrate pathway were significantly up-regulated when cells were cultivated in CGXII minimal medium with 1,000 mg/L propionate. The genes involved in the methylmalonyl-CoA pathway were up-regulated as well, but not as strongly as the 2-methylcitrate pathway (FIG. 3, panel a). C. glutamicum might prefer 2-methylcitrate pathway more than methylmalonyl-CoA pathway because the Gibbs free energy (ΔrG′m) of the reaction that converts propionyl-CoA to 2-methylcitrate is negative (ΔrG′m=−40.4±6.6 kj/mol), whereas that for conversion of propionyl-CoA to methylmalonyl-CoA is positive (ΔrG′m=−5±10.3 kj/mol) and need one ATP to fix CO2 (FIG. 3, panel a). To prove this hypothesis, the wild-type strain was cultivated in CGXII minimal medium with [¹³C3] Propionate. Analysis of pathway intermediates using LC-MS showed that more than 99% of [¹³C3] Propionate is transformed through the 2-methylcitrate pathway rather than through the methylmalonyl-CoA pathway (Extended Data FIG. S4a). All the above results indicated that the 2-methylcitrate pathway is favored over the methylmalonyl-CoA pathway to consume propionyl-CoA. To clarify whether this pathway causes growth inhibition, six genes (cgl0657-0659, prpD2B2C2 and cgl0694-0696, prpD1B1BC1) involved in the 2-methylcitrate pathway were deleted in the wild-type strain, resulting in strain Cz05, which only retains the methylmalonyl-CoA pathway for propionyl-CoA assimilation. In CGXII minimal medium containing 1,000 mg/L propionate, Cz05 had an even longer lag phase but showed less accumulation of methylcitrate than wild-type (FIG. 3, panel b), indicating that 2-methylcitrate accumulation is not the cause of growth lag, and further supporting the hypothesis that growth lag is caused by accumulation of propionyl-CoA and methylmalonyl-CoA, “dead-end” CoAs with no role in primary metabolism.

We performed additional experiments to show that accumulation of these CoAs is the cause of growth inhibition in propionate. We overexpressed the gene encoding E. coli propionyl-CoA synthetase (prpE) in Cz05 to generate Cz07, the genes encoding propionyl-CoA carboxylase (PCCase) from S. coelicolor to generate Cz08, or both of these heterologous genes to generate Cz09. Cz07, Cz08, and Cz09 grew slower than Cz05 (FIG. 3, panel d), indicating that enhanced methylmalonyl-CoA pathway would inhibit cell growth in propionate medium. To clarify which CoA metabolite contributes to growth inhibition, propionyl-CoA and methylmalonyl-CoA concentrations were measured in all strains. Compared to Cz05, both propionyl-CoA and methylmalonyl-CoA concentration were significantly increased in Cz07, Cz08 and Cz09 (FIG. 2, panel d), which indicated that both propionyl-CoA and methylmalonyl-CoA contribute to growth inhibition.

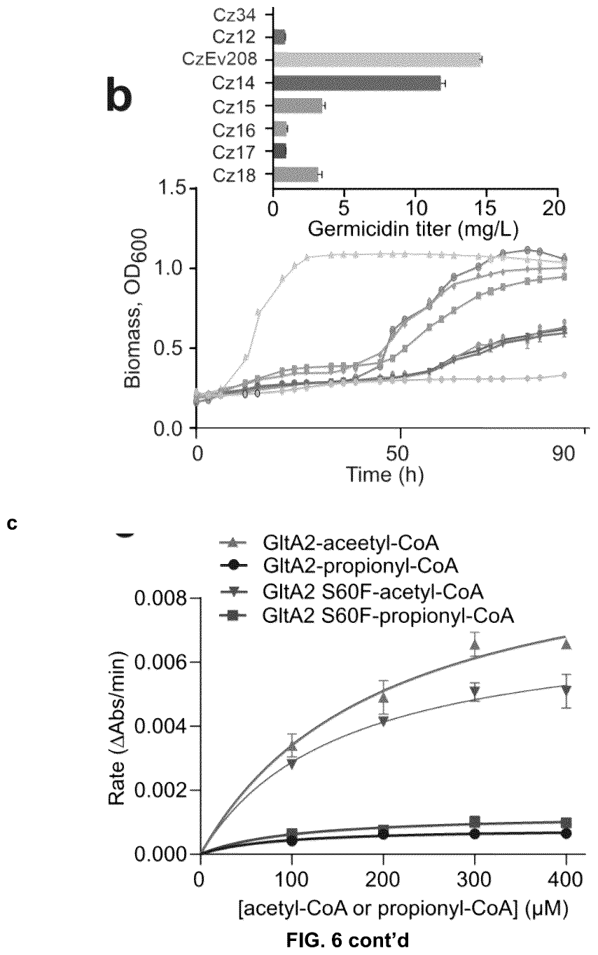

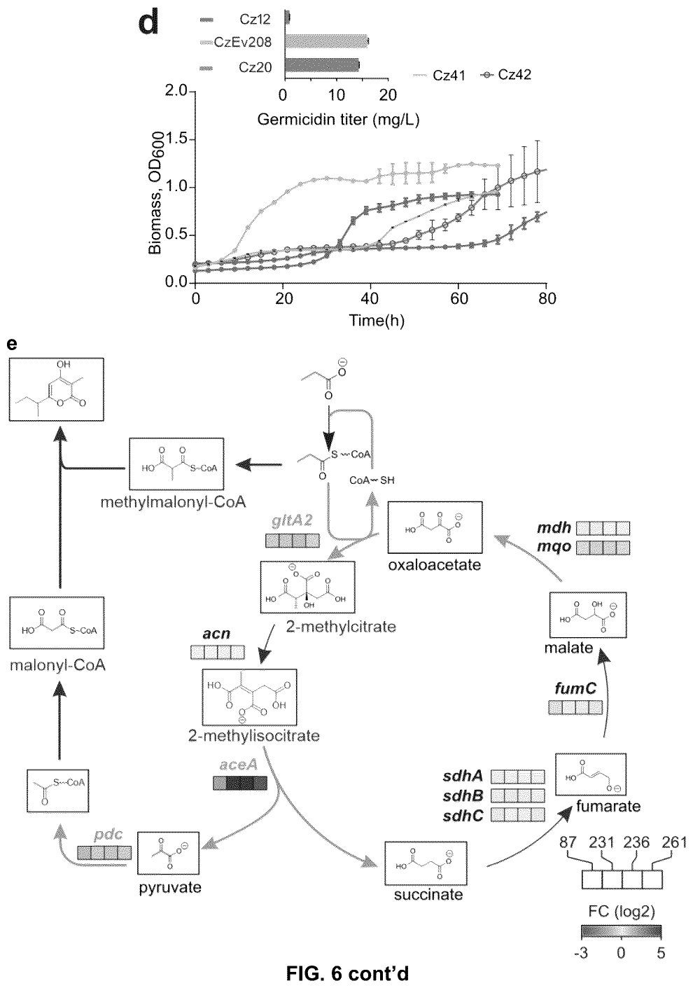

Further support for our CoA derivatives accumulation hypothesis comes from the fact that addition of vitamin B12 relieves growth inhibition in propionate-containing medium (Botella et al. 2009). C. glutamicum harbors a vitamin B12 dependent methylmalonyl-CoA/succinyl-CoA mutase (encoded by cgl1529-1530), which can catalyze the reversible conversion of these two metabolites (FIG. 3, panel a). Vitamin B12 addition provides a new outlet for converting “dead-end” methylmalonyl-CoA to succinyl-CoA, which then goes into the TCA cycle to support cell growth and regenerate free CoA-SH26. Transcriptomic analysis showed that these genes are upregulated in the presence of propionate compared to glucose only (FIG. 3, panel a). We deleted mcmAB (cgl1529-1530) in wild-type to generate strain Cz04, with its 2-methylcitrate pathway still intact. Compared to wild-type, little or no growth inhibition coupled with higher methylcitrate concentration were observed in Cz04 when it was cultivated in CGXII minimal medium with 1,000 mg/L propionate (FIG. 3, panel b), which provides further support that 2-methylcitrate accumulation does not contribute to growth inhibition in propionate medium. To confirm that vitamin B12 rescues growth by enabling conversion of methylmalonyl-CoA to succinyl-CoA, cgl1529-1530 were deleted in Cz05 to generate Cz06 (with both methylcitrate and methylmalonyl pathways deleted). We compared the growth of Cz04, Cz05, Cz06 and wild-type cells in CGXII with 1,000 mg/L propionate and 10 μM vitamin B12. Results showed that Cz05 grew much faster than Cz06 (FIG. 3, panel c) and had no lag phase compared with wild-type, which showed conclusively that enhancing methylmalonyl-CoA conversion to succinyl-CoA rescues cell growth in propionate medium. When we consider our results from evaluating (I) and (II) (FIG. 3, panel d), we reasoned that free CoA was fixed by propionate in (I) and is released when succinyl-CoA goes into TCA cycle in (II), which indicated that free CoA may be important to rescue growth in propionate medium. Compared to wild-type, free CoA concentration decreased in Cz05 and Cz06 (Extended Data FIG. S5), in addition, transcriptomic analysis showed that genes involved in de novo biosynthesis of free CoA were upregulated in propionate-containing medium (Extended Data FIG. 6c). To test whether free CoA is involved in propionate inhibition, the genes encoding the free CoA production pathway (aspartase from E. coli and aspartate 1-decarboxylase from C. glutamicum) were overexpressed to generate strain Cz10. Compared with Cz05, Cz10 showed a much shorter lag phase that was similar to wild-type (FIG. 3, panel e), which further supports that the lack of free CoA is one reason for the growth inhibition in propionate. The above results showed that enhance methylmalonyl-CoA conversion ion or CoA supplement would rescue cell growth in propionate, in order to clarify which approach works better, Cz05 was cultivated in different concentration of propionate with vitamin B12 or pantothenic sodium (precursor for free CoA biosynthesis). Results showed that although enough pantothenic sodium was added, the lag phase increased with increasing propionate concentration. And the lag phase in sodium pantothenate was always longer than when vitamin B12 was supplemented to the medium. These results showed that compared to CoA supplement, vitamin B12 works better to rescue cell growth in propionate medium by conversion methylmalonyl-CoA (Extended Data FIG. S6a, b, c, and d). Taken together, these results showed that accumulation of propionyl-CoA and methylmalonyl-CoA is the cause of growth inhibition in propionate medium, and that methylmalonyl-CoA conversion can rescue cell growth.

### 3. Methylmalonyl-CoA Dependent PKSs Rescue Cell Growth in Propionate Medium

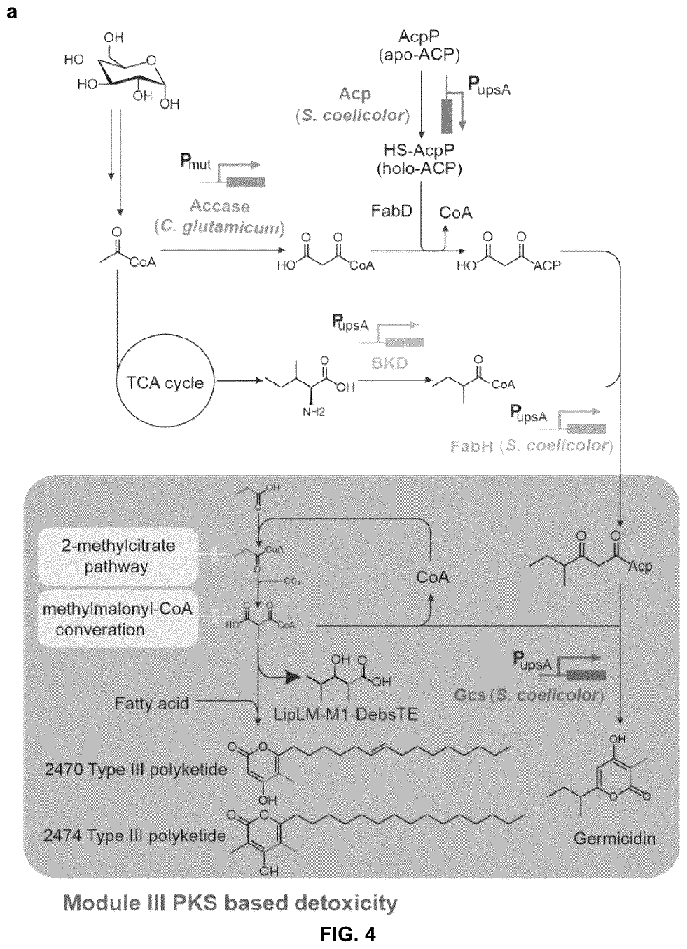

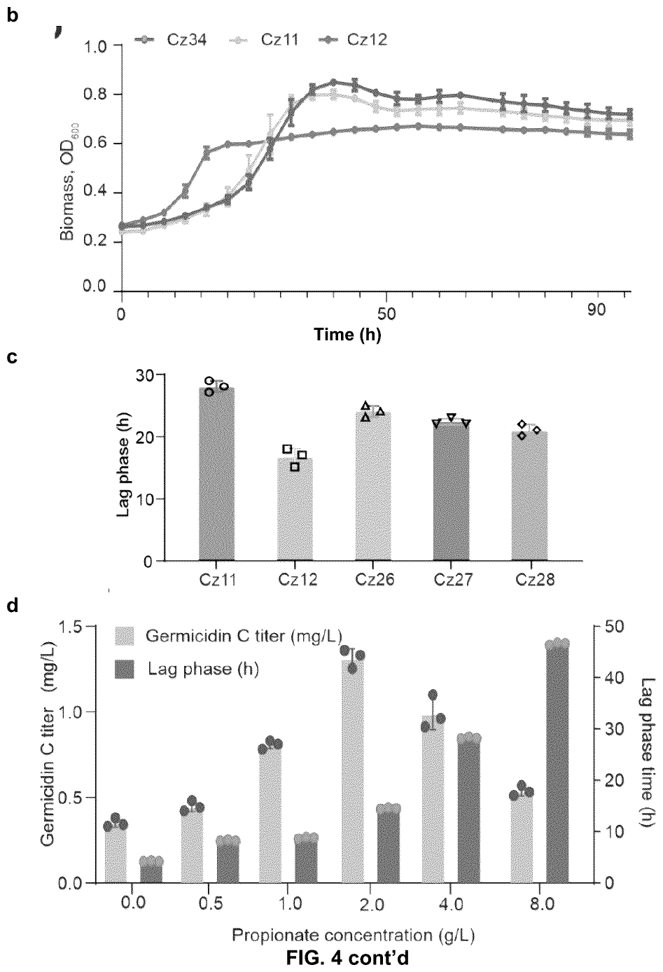

Since methylmalonyl-CoA is one of the most common extender units for PKSs11 and propionyl-CoA also acts as an important starter unit for PKSs34, increasing methylmalonyl-CoA or propionyl-CoA concentrations is a common strategy to promote polyketide production (Gonzalez-Garcia et al. 2020). Our previous results indicated that both propionyl-CoA and methylmalonyl-CoA are involved in propionate inhibition and conversion of methylmalonyl-CoA rescues cell growth in propionate medium. Taking all these results into consideration, we hypothesized that methylmalonyl-CoA conversion could be achieved by introducing a methylmalonyl-CoA dependent PKSs, which will not only rescue cell growth in propionate but also would promote polyketide production. Based on this hypothesis, we introduced several heterologous methylmalonyl-CoA dependent PKSs (FIG. 4, panel a).

Several genetic modifications were made to facilitate polyketide production. Phosphopantetheinyl transferases (PPTase) are required to activate acyl carrier protein (ACP) domains within PKSs. C. glutamicum encodes a native PPTase, so we first evaluated if heterologous expression of an additional PPTase, sfp from Bacillus subtilis, is necessary to support the native enzyme. Non-ribosomal peptide synthetase (NRPS) BpsA is an enzyme that produces a blue pigment, indigoidine (Pang et al. 2020), when its carrier protein domains are phosphopantetheinylated. We overexpressed bpsA and showed that coexpression of sfp resulted in higher indigoidine production compared to the strain lacking sfp (Extended Data FIG. S7a). Based on these results, we moved forward with strains that overexpressed sfp. We then intentionally increased the accumulation of methylmalonyl-CoA by deleting the 2-methylcitrate pathway (main pathway consuming propionyl-CoA) and cgl1529-1530 (mutase) to generate Cz34 strain. We used this as the base strain for subsequent PKS pathways integration.

The first PKS we tested was germicidin synthase (Gcs), a type III PKS from Streptomyces coelicolor, which uses methylmalonyl-CoA as the extension unit to synthesize germicidin (Chemler et al. 2012). This compound has significant inhibitory activity on hexokinase (HK2, IC50=0.78 mg ml-1), which is expressed at a high level in cancer cells³⁶. To test whether introduction of a methylmalonyl-CoA dependent PKS can rescue cell growth in propionate medium, the whole germicidin production pathway was introduced into Cz34 to generate Cz12 strain (FIG. 4, panel a). We compared the growth of Cz34 and Cz12 in CGXII minimal medium with 600 mg/L propionate. We found that Cz12 had a shorter lag phase (decreased 75%) compared to Cz34 when grown in propionate-containing medium (FIG. 4, panel b). Germicidin C production with [¹³C3] Propionate in our engineered strain (Extended Data FIG. S8) supported our hypothesis that the introduced germicidin production pathway can improve cell growth in propionate medium by converting methylmalonyl-CoA into a targeted product and releasing CoA-SH.

To show that this is a general phenomenon to all methylmalonyl-CoA dependent synthases, we introduced several other heterologous PKSs into Cz34, including two type III PKSs from Mycobacterium marinum (Mmar_2470 and Mmar_2474) (Parvez et al. 2018), and an engineered type I PKS, LipLM-M1-debsM6TE, which we have shown to produce hydroxy acids in Streptomyces (Yuzawa et al. 2013). All of the heterologous PKS pathways resulted in a shorter lag phase in 600 mg/L propionate compared to Cz34 lacking a PKS pathway, although the degree of growth improvement varied between PKS pathways (FIG. 4, panel c, Extended Data FIGS. S7, 9, and 10).

In order to further increase germicidin titer, we cultivated the engineered strain in a minimal medium with different propionate concentration and measured growth and germicidin production. Germicidin production increased as the propionate concentration increased, even though the lag phase also increased especially when the propionate concentration over 1,000 mg/L (FIG. 4, panel d and Extended Data FIG. S11). Although introduction of the germicidin PKS improved cell growth in propionate medium, the engineered strains still grew slower in propionate medium compared with cells growing without propionate. This inhibition was more severe when propionate concentration increased beyond 1,000 mg/L.

### 4. Methylmalonyl-CoA Toxicity Promote Germicidin Production Pathway Evolution

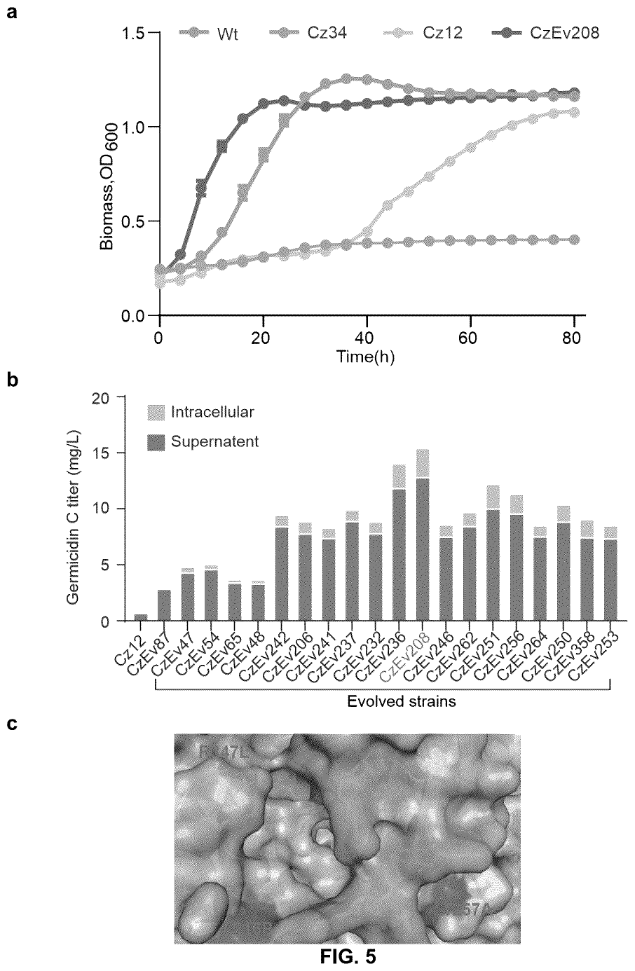

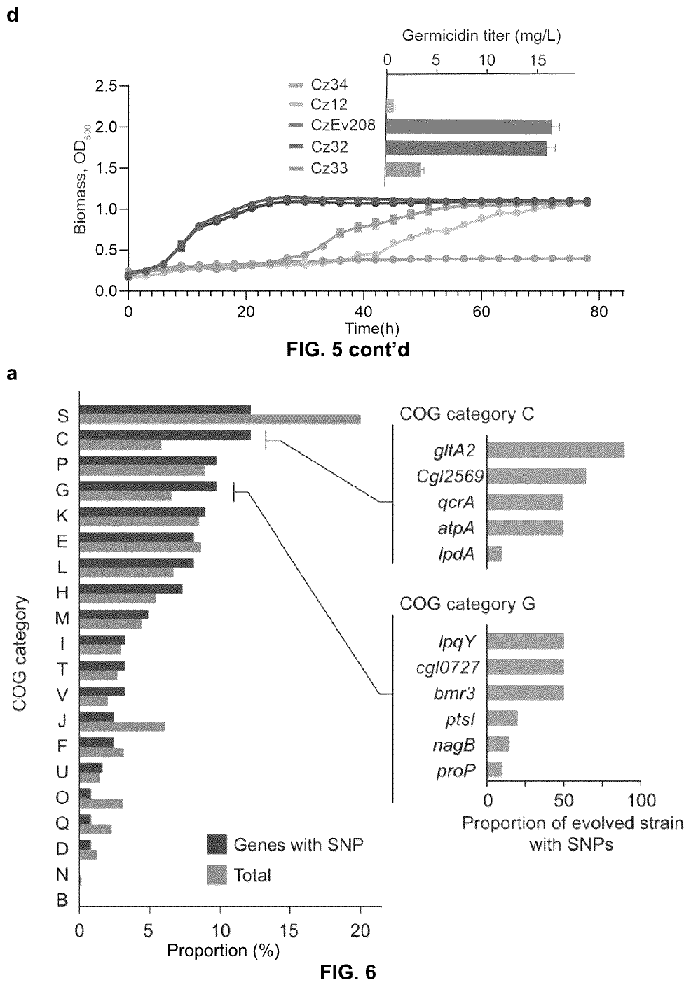

Propionate tolerance was first evaluated using Cz034 in CGXII minimal medium to evaluate the highest propionate concentration for ALE. The lag phase increased as the propionate concentration increased, and no growth was observed when the propionate concentration was over 8,000 mg/L (Extended Data FIG. S11a, b). Then, ALE was applied to improve propionate tolerance and germicidin production in Cz12 by repeatedly growing the strains in increasing concentrations of propionate until we reached 8,000 mg/L (Extended Data FIG. S12). After two months of evolution, 563 single colonies were picked up from plates. Most of them grew faster than the initial strain (Extended Data FIG. S13a, b, c, d). Surprisingly, evolved strain CzEv208 (knockout methylcitrate pathway and methylmalonyl-CoA pathway) grew even faster in 8,000 mg/L propionate than wild-type growing in CGXII minimal medium without propionate (FIG. 5, panel a). Compared to Cz12, the growth rate increased more than 140%, and the lag phase also significantly decreased (decreased by 80%) in CzEv208 (FIG. 4, panel d and Extended Data FIG. S13e, f). All of these results indicate that methylmalonyl-CoA utilization ability in CzEv208 was significantly improved. To clarify whether the increased germicidin production improved methylmalonyl-CoA utilization, germicidin titer was measured on evolved strains. Compared to Cz12, germicidin production in almost all evolved strains was significantly increased (FIG. 5, panel b). Especially, germicidin titer increased more than 18-fold in CzEv208 (13.6±0.05 mg/L) compared with Cz12 (0.78±0.03 mg/L) (FIG. 5, panel d), indicating that strains harboring beneficial mutations for germicidin production have been selected during ALE.

To determine which genetic changes in evolved strains are correlated with improved titers and growth rates, we first sequenced all six genes involved in the germicidin production pathway in the best performing evolved strains. Three promising mutations (L216P, T257A and R347L) were found in Gcs of evolved strains (4 out of 6 evolved strains). To identify the locations of the mutated residues, SWISS-Model was used to model the structure of Gcs based on 3v7i.1.A (PDB) (Chemler et al. 2012). Two mutations (L216P and T257A) are adjacent to the substrate binding tunnel (cyan, Arg (276/277/280/317)), and one mutation (R347L) is close to the active site (green, Cys175-His312-Asn346) (FIG. 5, panel c) 35. In order to validate whether these mutations were responsible for the improved germicidin production and cell growth in evolved strains, we replaced the native gcs gene in Cz12 with different mutant versions of the gene found in our evolved strains. Neither L261P nor T257A (singly or in combination) affected cell growth and germicidin production (Extended Data FIGS. S14a and b). However, when Gcs R347L was introduced into Cz12 to generate Cz33, we found that the lag phase decreased 32.5% and germicidin titer increased 337.5% compared to Cz12 (FIG. 5, panel d). Conversely, when the mutated gcs gene was replaced by the native gcs gene in CzEv208 to generate Cz32, germicidin titer decreased 18%, although lag phase was not significantly different in the two strains (FIG. 5, panel d). These results showed that we can use propionate as a selective pressure to induce beneficial mutations in a methylmalonyl-CoA-dependent PKSs.

### 5. Evolved Citrate Synthase (GltA2)-Based Methylcitrate Pathway Rescue Cell Growth by Consuming Propionyl-CoA

Although replacing native Gcs of Cz12 to evolved Gcs improved cell growth in propionate, the lag phase was still longer than that of evolved strain, indicating that some other mutations may be involved in rescuing cell growth in evolved strain. In order to further clarify the mechanism, genome sequencing was performed on 19 evolved strains and we observed many other mutations in our evolved strains' genome that could lead to improved fitness in addition to those in the gcs gene itself. Clusters of orthologous groups (COG) category (Huerta-Cepas et al. 2019) of C. glutamicum genes were determined by using EggNog and distribution of categories in total and SNP containing genes were compared. For the genes with SNPs, the proportion of category C (energy production and conversion) and category G (carbohydrate transport and metabolism) was enriched than that of the total genes. Citrate synthase (GltA2), belongs to category C, was mutated in most of the evolved strains (18 out of 19 evolved strains) (FIG. 6, panel a). In order to test whether SNPs in GltA2 are involved in cell growth rescue and germicidin production increase, the mutated GltA2 in CzEv208 was replaced by the native GltA2 to generate Cz14. Compared to CzEv208, Cz14 grew much slower and germicidin production also decreased (FIG. 6, panel b). Next, we replaced native GltA2 with the evolved GltA2 variant in Cz12 to generate Cz15. Cz15 grew faster than Cz12 (lag phase decreased by 37%) and germicidin titer also increased (increased by 318%) (FIG. 6, panel b), indicating that the mutation that arose in GltA2 is involved in the improved growth phenotype and germicidin production. The evolved GltA2 had three mutations: E239G, R310C, and S60F (Extended Data FIG. S15a). These mutations were individually introduced into Cz12 to generate Cz16, Cz17 and Cz18. Compared to the other two mutation sites, S60F (Cz18) showed the greatest impact on cell growth in propionate medium (FIG. 6, panel b). Citrate synthase is a key component of the TCA cycle and glyoxylate cycle. The latter pathway is closely analogous to the methylcitrate pathway in that the substrates (acetyl-CoA and propionyl-CoA) and products (glyoxylate and pyruvate) differ by a single methyl group. We therefore hypothesized that the evolved mutations in GltA2 resulted in an improved ability to accept propionyl-CoA as a substrate. To directly test this, native GltA2 and GltA2 S60F were purified (Extended Data FIG. S15b). In vitro citrate synthase kinetic assays showed that the evolved GltA2 S60F had higher activity with propionyl-CoA compared to native GltA2 (FIG. 6, panel c). Transcriptomic data showed that the genes encoding isocitrate lyase (aceA) and malate synthase (aceB) were upregulated in our evolved strains (CzEv231, CzEv236, CzEv261) compared to initial strain Cz12. This led to the hypothesis that mutation of GltA2 S60F to accept propionyl-CoA as a substrate, and overexpression of the native aceA genes, essentially led to evolution of a new methylcitrate cycle in C. glutamicum from its endogenous glyoxylate cycle (FIG. 6, panel e). In order to test whether the evolved methylcitrate pathway involved in propionate rescue, we deleted aceA and aceB in CzEv208 to generate Cz20. Compared to CzEv208, Cz20 had a longer lag phase, and germicidin titer also decreased (FIG. 6, panel d). In addition, LC-MS results also showed that the mass of labeled germicidin C matched the hypothesis (Extended Data FIG. S16), which means the evolved 2-methylcitrate pathway involved in cell growth rescue in propionate medium. In addition to GltA2, the COG analysis results also showed that category G (carbohydrate metabolism and transport) was enriched among SNP containing genes. Among SNP containing genes in category G, knockout bmr3 (cgl0380), permeases of major facilitator superfamily (transporter), in CzEv208 to generate Cz41 significantly increased lag phase. In addition, overexpression of evolved bmr3 in Cz12 to generate Cz42 rescued cell growth in propionate medium (FIG. 6, panel d), indicating that bmr3 membrane protein is also involved in propionate rescue.

### DISCUSSION

Our study revealed that addition of propionate to minimal media leads to inhibitory levels of propionyl and methylmalonyl-CoA in C. glutamicum, and demonstrated that we can leverage this growth inhibition as a selective pressure to evolve strains with improved growth and polyketide production. We expressed several different PKS systems in C. glutamicum, and in all cases polyketide production was correlated with faster growth in propionate, demonstrating that this strategy can be applied generally.

We used germicidin synthase as an example to demonstrate the utility of this system for improving polyketide production through directed evolution. Some of the strains showing improved growth harbored beneficial mutations in the PKS gene that improved the activity of the enzyme. However, our results showed that mutations arising elsewhere in the genome were much more common than mutations in the PKS gene itself. The reason may be that compared to genomic DNA, PKSs are too small to introduce mutations or multi resistance mechanisms need to be activated to deal with these stresses. For example, although gltA2 and prpC showed significant sequence similarities, gltA2 did not show methylcitrate synthase activity in C. glutamicum (Claes et al. 2002). Here, we found that mutations in gltA2, which gave rise to a new metabolic pathway to catabolize propionyl-CoA based on the endogenous glyoxalate cycle, occurred in most of the evolved strains. Results showed that replacing native gltA2 with evolved gltA2 in Cz12 relieves growth inhibition in propionate medium, and that lag phase increases when the evolved gltA2 in CzEv208 was replaced by native gltA2 (FIG. 6, panel b). We performed in vitro citrate synthase assays to show that evolved GltA2 has higher activity with propionyl-CoA compared to native GltA2 (FIG. 6, panel c). All of these results indicated that the mutations occurred in gltA2 led to the emergence of an evolved methylcitrate pathway to rescue cell growth in propionate in which mutant GltA2 can now perform the same function as methylcitrate synthase. This novel pathway can then relieve propionate inhibition by consuming propionyl-CoA and release free CoA (FIG. 3, panel a), similar to the native methylcitrate pathway. [¹³C3] Propionate labeled carbon flux analysis already showed that more than 99% propionate goes into methylcitrate pathway in wild-type (Extended Data FIG. S2). However, based on LC-MS results, we found that the most abundant product germicidin C was only labeled by methylmalonyl-CoA (MS [H+H]+=186), not by both methylmalonyl-CoA and 2-methylbutyryl-CoA (MS [H+H]+=188) (Extended Data FIG. S16). This indicated that propionyl-CoA in CzEv208 was mainly converted to methylmalonyl-CoA not goes into the evolved methylcitrate pathway and the evolved methylcitrate pathway activity is not comparable to native methylcitrate pathway, which indicated that the evolved GltA2-based methylcitrate pathway not only rescue cell growth in propionate medium but also push more propionyl-CoA goes into methylmalonyl-CoA pathway than the native methylcitrate pathway.

Another example of a beneficial mutation that occurred outside the PKS gene was discovered in the gene bmr3 (cgl0380), which also relieved propionate inhibition. COG category analysis showed that bm3 belongs to category G, suggesting that bmr3 may encode a membrane protein that is involved in propionate transport. Blast results showed that Bmr3 is a permease belonging to the major facilitator superfamily, proteins which facilitate movement of small solutes across cell membranes in response to chemiosmotic gradients. In order to test this hypothesis, propionate consumption was measured in wild type, Cz12, CzEv208, Cz41 and Cz42. Results showed that compared to Cz12, propionate consumption in Cz42 increased (p=0.068>0.05), and compared to CzEv208, the propionate utilization in Cz41 decreased (p=0.068>0.05). But the propionate consumption did not show significant difference between these strains (Extended Data FIG. S18). Mutations in membrane proteins therefore represent another way for the cells to circumvent propionate based growth inhibition which should be accounted for in future engineering efforts. As described above, a vast majority of the mutations were not in the target PKS gene, future directed evolution experiments will employ strategies like introducing targeted mutagenesis systems (Yi et al. 2021) in which we intentionally increase the mutation rate in the PKS gene(s). In summary, we have provided a new system for growth-based screening for PKS activity and a general strategy for improving high-valuable products titers through directed evolution. Previous PKS engineering examples without selection pressure have focused on precursor utilization or low-throughput analytical chemistry based screening. The ability to screen and select improved polyketide producers by growing in propionate will greatly improve the ease and rate, which lead us study these complex biosynthetic enzymes.

### Methods and Materials

### Strain and Reagents

Plasmids and different strains are listed in Extended Data Table 1. Phusion polymerase and HiFi DNA Assembly kit were purchased from NEB, different restriction enzymes were purchased from Thermo Scientific. All oligonucleotides were synthesized at Integrated DNA Technologies (IDT). Germicidin C standard was synthesized in Wuxi ApTec (Tianjin). [¹³C3] Propionate sodium was purchased from Cambridge Isotope Laboratories (USA). Other chemicals were all purchased from Sigma-Aldrich. All codon optimized heterologous genes were synthesized by Genscript and are listed in Extended Data Note 1.

### Methods

Competent cell preparation and electrotransformation. i) C. glutamicum for preparation of competent cells were firstly cultivated in BHI (37 g Brain Heart Infusion powder in 1 L H₂O) liquid medium overnight (30° C., 200 rpm), then cells were transformed into EPO medium (37 g BHI, 25 g Glycine, 10 mL Tween, 4 g Isoniazid), 30° C., 200 rpm for 5-6 h, OD600 is around 1.0). ii) Transform cells into fresh 50 ml tubes, 4° C. 4000 g for 10 min. Discard supernatant and add 20 mL pre-cold 10% glycerol, resuspend pellets. iii) Repeat ii) three times. iv) add 1 mL 10% pre-cold 10% glycerol to resuspend pellets. v) take 80 μl competent cells into pre-cold electrotransformation cuvettes (0.2 cm gap), add 500 ng episomal plasmid. vi) 2500U, 25 μF, 200Ω (BioRAD PC Module). vii) 46° C. heat shock for 5 min. viii) 30° C. for 1-2 h. ix) put cells in BHI+Kan select plates for 2 days. x) pick up signal colonies and scratch in BHI+10% Sucrose plate, 30° C. for 1-2 days to loop out the selection marker. xi) pick up colonies and PCR test.

Colony PCR. Pick up colonies from plate and cultivate in BHI medium (30° C., 200 rpm) overnight (around 14-16 h). Prepare digestion solution (120 μL zymolase (E1005, ZYMO RESEARCH) and 2500 μL PBS pH=7.2) for 96 reactions. Take 3-5 μL overnight culture into 20 μL digestion solution, 30° C. for 1 h. Add 3-5 μL dimethyl sulfoxide (DMSO), then 98° C. for 10 min. Take 2 μL for 50 μL PCR reaction. Growth curve measurement. Various cells were pre-cultivated in BHI liquid medium (30° C., 200 rpm) overnight. Cells were transformed into CGXII minimal medium (20 μL culture into 2 mL medium), 30° C., 200 rpm for 16-18 h. Then cells were subcultivated into 48 deep well plate containing fresh CGXII minimal medium with or without propionate (original OD600 is around 0.1). Growth curve was measured by SpectraMaX M2e at 30° C.

[¹³C3] labeled experiments. For all experiments using [¹³C3] propionate assay, different strains were pre-cultivated in BHI liquid medium (30° C., 200 rpm) overnight. Then cells were cultivated into CGXII minimal medium (30° C., 200 rpm) for 8-12 h. Then cells were subcultivated in fresh CGXII minimal medium with 1 g/L [¹³C3] propionate (99.0% atom enrichment, Cambridge Isotope Laboratories, USA). For metabolites or CoA measurement, cells were collected during the early exponential phase (OD600 is around 0.8-1.2). For targeted chemical measurement, cells were collected at stationary phase.

Metabolites extraction and measurement. Different strains were collected during the early exponential phase. Culture was centrifuged at 4° C., 5000×g for 10 min. Cells pellets were stored at −80° C. (for long store). Cells (8 mg CDW of cells, approx. 2 mL of OD600=2 culture) and supernatant were quenched with acetonitrile/methanol/50 mM formic acid (45:45:10, v/v) containing 5 nM [13C] malonyl-CoA (internal standard) to a final volume of 1 mL. The extraction was performed on ice with intermittent vortexing for 15 min, followed by a 3 min centrifugation at 13,000×g and 4° C. Supernatant was freeze dried and metabolites were resuspended in 100 μL resuspension buffer (50 mM ammonium formate, pH 3.0, 2% methanol) 37. LC-MS was used to analyze metabolite profiles according to a previous method whose LC and MS conditions are described by Baidoo et al., 2019 and Kim et al., 2020, respectively (Kim et al. 2021; Baidoo et al. 2019).

LC-MS to measure Germicidin C production. 0.5 mL culture was centrifuged at 13,000×g 4° C. for 10 min. Add 200 μL methanol into 200 μL supernatant. Samples were filtered using 3 kDa plate (PALL, Omega 3K), 5000×g for 1 h at 4° C. Cell pellets was resuspended using 0.5 mL methanol, then samples were cultivated in Eppendorf Thermomixer R Mixer (23° C., 1000 rpm) overnight (around 12-14 h). 0.5 mL ddH₂O was added into samples and all samples were filtered using 3 kDa plate (PALL, Omega 3K). LC-MS analysis were performed on the LC-MSD iQ system with Mass selective detector (Agilent). 2 μL of different samples were loaded onto the Kinete®XB-C18 column (2.6 μm, 100×3 mm, Phenomenex) at a flow rate of 0.425 mL/min at 45° C.: 0 to 5.0 min, 80% mobile phase A (0.1% formic acid in water)/20% mobile B (0.1% formic acid in methanol); 5.0 min to 6.0 min, gradient from 51.8% mobile phase A/48.2% mobile B to 5.0% mobile phase A/95% mobile B; 6.0 min to 9.0 min, 5% mobile phase A/95% mobile B; 9.0 min to 9.1 min, gradient from 5% mobile phase A/95% mobile B to 80% mobile phase A/20% mobile B. 9.1 min to 12.0 min, 80% mobile phase A/20% mobile B.

HPLC to measure propionate utilization. Culture was centrifuged 13,000×g for 10 min at 4° C. 100 μL supernatant was added into 100 μL 5 mM H₂SO₄. Samples were filtered using 3 kDa plate (PALL, Omega 3K). HPLC analysis were performed on the HPLC (Agilent 1200 series). 5 μL of different samples were loaded onto the using HPX-87H (Bio-Rad) at a flow rate of 0.5 mL/min at 65° C. Diode array detector (DAD) and refractive index detector (RID) were used to detect propionate.

Protein purification. pET-LIC-Cg12569, pET-LIC-GltA2 wild-type and pET-LIC-GltA2 mutation were transformed into BL21 (DE3) competent cells following the protocol. i) ingle colonies were picked up from LB+Kan plates and cultivated in 5 mL LB+Kan liquid medium overnight at 37° C., 200 rpm. ii) 2 mL overnight culture was inoculated in fresh 500 mL Terrific Broth+4% glycerol liquid medium (6 g tryptone, 12 g yeast extract, 20 mL glycerol with 500 mL H2O) in a 2 L baffled culture flask, grew at 37° C., 200 rpm until OD600 reached 0.6-1.0. iii) The flask was transferred to 20° C. with shaking for 1 h before 0.1 mM IPTG induction. iv) The cells were harvested after 18-20 h incubation at 20° C., 200 rpm. v) Culture was centrifuged at 4° C., 4,000 rpm for 30 min, pellet cells and freeze cells. vi) re-suspend cells in 50 mL Lysis Buffer (300 mM NaCl, 25 mM Tris pH 7.5 and 10% glycerol). vortex about 30 min at 25° C., then place on ice and make sure pellet is completely homogenized. vii) sonicate (30 s on then 60 s off, repeat for 6 times). viii) 17000 rpm centrifugation for at least 30 min, followed by 0.45 μm membrane filtration to collect supernatants. Ni affinity chromatography was applied for protein purification. Filtered supernatants were loaded onto a 5 mL His-Trap column at 3 mL/min flow rate, followed by 50 mL buffer A (300 mM NaCl, 25 mM Tris pH 7.5, 20 mM imidazole pH 7.5, and 10% glycerol) wash at 4 mL/min flow rate. The His-tagged protein was eluted with buffer B (300 mM NaCl, 25 mM Tris pH 7.5, 500 mM imidazole pH 7.5, and 10% glycerol) at 4 mL/min flow rate and 30 mM/min imidazole gradient. Collected fractions were analyzed by SDS-PAGE, confirming >90% homogeneity. Fractions containing the target protein were dialyzed in 1 L storage buffer (100 mM NaCl, 25 mM Tris pH 7.5 and 10% glycerol) at 4° C. overnight, concentrated to 3.0 mg/mL final protein concentration, and stored at −80° C.

In vitro assay to measure Cg2569 activity. All samples were run in duplicate. Prepare different reaction stock solutions (acetate, propionate: 100 mM, succinyl-CoA, propionyl-CoA, acetyl-CoA and free CoA: 10 mM). Take 0.2 mM succinyl-CoA, 0.2 mM propionate and 2 μm purified Cg2569 protein, PBS (pH=7.4) was added to 100 μL. Incubate at room temperature for 20 min38. Then 100 μL methanol was added into the reaction system, spin down at 4° C. Take the solution into 3K column, 13,000×g for 30 min at 4° C. Take 50 μL for LC-MS measurement (A common approach for absolute quantification of short chain CoA thioesters in prokaryotic and eukaryotic microbes).

In vitro assay to measure Citrate synthase activity. All samples were run in duplicate. Following the citrate synthase activity assay kit protocol (Catalog Number MAK193, SIGMA-ALDRICH) and made some modification. 5 μmol purified citrate synthase was added into reaction system, which containing 6.4 μl 50 mM DNTB, 10 μl 20 mM oxalacetate and different concentration of acetyl-CoA and propionyl-CoA (0 μM, 100 μM, 200 μM, 300 μM, 400 μl and 500 μM), assay buffer was added to 200 μl. Incubate 96 well plate at 25° C. for 20 min. Measure the absorbance at 412 nm using SPMAX-384PLUS plate reader every 9 s.

Genome re-sequence and data analysis. Different strains were cultivated in BHI liquid medium overnight (30° C., 200 rpm). 20 μl culture was transferred into CGXII minimal medium (30° C., 200 rpm) for 16 h. Then 20 μl culture was transferred into CGXII minimal medium with 8 g/L propionate medium (30° C., 200 rpm) for 32 h or 120 h for initial strains. Pellet cells and stored in −80° C. Genomic DNA was isolated followed the protocol (Wizard® Genomic DNA Purification Kit). 100 ng of gDNA was sheared to 600 bp using the fragmentation enzyme in xGen™ DNA Lib Prep EZ kit (Integrated DNA Technologies, Inc) and size selected using SPRI beads (Beckman Coulter). The fragments were treated with end-repair, A-tailing, and ligation of Illumina compatible adapters (Integrated DNA Technologies, Inc) using the xGen™ DNA Lib Prep EZ kit. Bioanalyzer High Sensitivity DNA Kit (Agilent) and Qubit Fluorometers (ThermoFisher Scientific) were used to determine the concentration of the libraries. Libraries were sequenced on the Illumina Miseq.

Transcriptome profiling. Different strains were cultivated in BHI liquid medium overnight (30° C., 200 rpm). 20 μl culture was transferred into CGXII minimal medium (30° C., 200 rpm) for 16 h. Then 20 μl culture was transferred into CGXII minimal medium with 8 g/L propionate medium (30° C., 200 rpm) for 12 h (OD 600 is around 0.8) or 70 h (OD 600 is around 0.8) for initial strains. Total RNA was extracted by GENEWIZ Company. The raw reads from each sample and analysis of differential expression were completed and provided by GENEWIZ Company. GO enrichment analysis was performed using the Platform for Integrative Analysis of Omics (PIANO) R package. COG term information was obtained from the EggNog. Differential expression levels (log 2-fold change) and corresponding significance levels (P values) of genes obtained by comparing evolved strains with initial strain were used as inputs. COG terms were calculated and scored by modulation of the expression level of genes within the same COG term.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified host cell reduced for expression of one of more endogenous enzyme(s) which enable the catabolism of propionyl-CoA and/or methylmalonyl-CoA, and comprising a methylmalonyl-CoA-dependent polyketide synthase (PKS).

2. The genetically modified host cell of claim 1, wherein the genetically modified host cell is a genetically modified Corynebacterium cell.

3. The genetically modified host cell of claim 2, wherein the genetically modified Corynebacterium cell is a Corynebacterium glutamicum.

4. The genetically modified host cell of claim 1, wherein the methylmalonyl-CoA-dependent PKS is a hybrid PKS comprising a loading domain capable of loading methylmalonyl-CoA as a starting substrate.

5. The genetically modified host cell of claim 4, wherein the loading domain capable of loading methylmalonyl-CoA as a starting substrate is a loading domain of germicidin PKS.

6. The genetically modified host cell of claim 1, wherein endogenous genes PrpC, PrpD, PrpB, MutaseA, or MutaseB are deleted or knocked out.

7. The genetically modified host cell of claim 1, wherein the genetically modified host cell is overexpressed for AcpA and/or FabH.

8. A method of producing a polyketide using methylmalonyl-CoA as a substrate, said method comprising: (a) providing a genetically modified host cell of claim 1, and (b) culturing or growing the genetically modified host cell such that the genetically modified host cell expresses the methylmalonyl-CoA-dependent PKS and produces a polyketide using methylmalonyl-CoA as substrate.

9. The method of claim 8, wherein the (b) culturing or growing step comprises providing propionate to the genetically modified host cell.
